(19) United States
(12) Patent Application Publication
(10) Pub. No.: US 2015/0147105 A1
(43) Pub. Date: May 28, 2015

(54) SOLID ANTIPERSPIRANT COMPOSITION WITH AN ENHANCED EVAPORATIVE COMPONENT

(71) Applicant: The Dial Corporation, Scottsdale, AZ (US)

(72) Inventors: Travis T. Yarlagadda, Lenexa, KS (US); Aleidatje M. Lester, Phoenix, AZ (US); Debra K. Butterworth, Scottsdale, AZ (US)

(73) Assignee: The Dial Corporation, Scottsdale, AZ (US)

(21) Appl. No.: 14/087,232

(22) Filed: Nov. 22, 2013

(51) Int. Cl. A61K 8/11; A61Q 15/00

(52) U.S. Cl. CPC A61K 8/11; A61Q 15/00
USPC 401/95; 424/65; 424/401

(57) ABSTRACT

Materials and apparatus are provided that include an enhanced evaporative component. A solid antiperspirant composition includes an antiperspirant agent disposed within the solid antiperspirant composition. Further, the solid antiperspirant composition includes an enhanced evaporative component disposed within the solid antiperspirant composition. The enhanced evaporative component includes a first spreading agent configured to reduce the surface tension of perspiration molecules. The enhanced evaporative component also includes a volatile to increase the volatility of perspiration molecules. Still further, the enhanced evaporative component includes an entrapment material disposed within the solid antiperspirant composition to contain the volatile.

### FIELD OF THE INVENTION

The present invention generally relates to enhancing evaporation of perspiration molecules and providing an increased perception of dryness, and more particularly relates to a solid antiperspirant composition with an enhanced evaporative component.

### BACKGROUND OF THE INVENTION

Antiperspirants are personal care products that are used to prevent or reduce perspiration and odors resulting from perspiration, particularly perspiration from a person's underarm. Perspiration also forms wet areas on the affected skin, i.e., a person's underarm, and on clothing proximate to the person's underarm. Such perspiration, odors, and wetness can be uncomfortable and embarrassing.

Accordingly, it is desirable to have an antiperspirant composition that prevents or reduces perspiration wetness and perspiration odors. Additionally, it is desirable to have an antiperspirant composition that is comfortable to the individual and that delivers a perception of wetness protection. Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A solid antiperspirant composition with an enhanced evaporative component includes an antiperspirant agent disposed within the solid antiperspirant composition. The composition also includes an enhanced evaporative component disposed within the solid antiperspirant composition. The enhanced evaporative component includes a first spreading agent configured to reduce the surface tension of perspiration molecules. The enhanced evaporative component also includes a volatile configured to increase the volatility of perspiration molecules. The enhanced evaporative component also includes an entrapment material to contain the volatile.

An antiperspirant product with an enhanced evaporative component includes a container. The product also includes a solid antiperspirant composition housed within the container. The solid antiperspirant composition includes an antiperspirant agent. The solid antiperspirant composition also includes an enhanced evaporative component that includes a spreading agent configured to reduce the surface tension of perspiration molecules. The enhanced evaporative component also includes a volatile configured to increase the volatility of perspiration molecules. The enhanced evaporative component also includes an entrapment material to contain the volatile.

A solid antiperspirant composition with an enhanced evaporative component includes an active ingredient. The active ingredient includes an antiperspirant agent to reduce perspiration, perspiration odors, perspiration wetness, or combinations thereof. The solid antiperspirant composition also includes a passive ingredient. The passive ingredient includes an enhanced evaporative component with a spreading agent that forms 0.1 to 10.0 weight percent of the enhanced evaporative component. The enhanced evaporative component also includes a volatile forming 40.0 to 60.0 weight percent of the enhanced evaporative component. The enhanced evaporative component also includes an entrapment material to contain the volatile.

### BRIEF DESCRIPTION OF THE DRAWINGS

The present invention will hereinafter be described in conjunction with the following drawing figures, wherein like numerals denote like elements, and

FIG. 1 is a diagram of an example of a solid antiperspirant composition with an enhanced evaporative component according to the principles described herein.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

As described above, perspiration is a common occurrence in most individual's lives. Perspiration itself and odors emanating from the perspiration can be both uncomfortable and embarrassing. For example, the perspiration may make skin sticky and warm to the touch, which may be uncomfortable. Also, the odor from the perspiration may be embarrassing to the individual, in a public setting for example. Additionally, perspiration often produces wetness at the location of perspiration as well as on clothing proximate to the perspiration. Such wet spots may be uncomfortable as they may lead to chafing. Additionally, the wet spots may prove unsightly and embarrassing for an individual.

The principles described herein provide a composition for enhancing the evaporation of perspiration molecules. In other words, the solid antiperspirant composition may exhibit enhanced wetness protection. In addition, the solid antiperspirant composition may reduce a perception of perspiration by a user. This perceived wetness protection by an individual may contribute to a satisfactory consumer experience. Accordingly, the principles described herein describe a composition for enhancing the actual effects of an antiperspirant compound as well as delivering a perceived sensation of wetness protection via an enhanced evaporative component. Such an enhanced evaporative component may include a spreading agent to reduce the surface tension of perspiration molecules in perspiration. The reduction in surface tension may cause the water droplets of the perspiration molecules to occupy a greater surface area, which in turn improves the evaporative process of perspiration reduction. The spreading agent may be 0.1 to 1.0 weight percent of an enhanced evaporative component.

In some examples, other ingredients may be included in the enhanced evaporative component of the solid antiperspirant composition, such as a volatile. The volatile may evaporate next to the user's skin when released from an encapsulation material, which may deliver a dry “feel” to the user, or in other words, may deliver an increased perception of wetness protection resulting in a more satisfactory consumer experience. The volatile may be 40.0 to 60.0 percent weight of the enhanced evaporative component.

An enhancement of the evaporative mechanism may result in an enhanced actual wetness protection as well as an enhanced perception of wetness protection. Enhancements which may protect against perspiration wetness, reduce perspiration odors, provide comfort to the individual, and leave the consumer with an overall satisfactory experience.

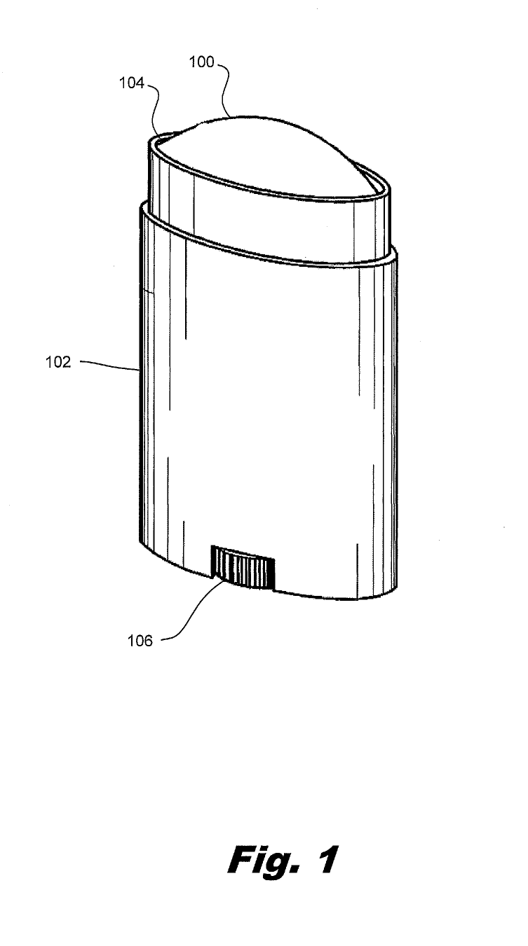

FIG. 1 is a diagram of an example of a solid antiperspirant composition (100) with an enhanced evaporative component according to the principles described herein. In this example, the solid antiperspirant composition (100) is held within a container (102) that has an opening (104) that allows the solid antiperspirant composition (100) to be pushed out of the container (102) and onto a surface. For example, the solid antiperspirant composition (100) may extend beyond the opening (104) of the container (102) and be applied to skin. As indicated in FIG. 1, the solid antiperspirant composition (100) may be approximately dome-shaped and may be applied to skin. More specifically, the solid antiperspirant composition (100) may be applied to an underarm. The container (102) may also include a dispenser to dispense the solid antiperspirant composition (100). For example, the solid antiperspirant composition (100) can be pushed out of the opening (104) by rotating a dial (106), or a knob, positioned at the bottom of the container (102). As the dial (106) is rotated, a platform internal to the container (102) moves up and pushes the solid antiperspirant composition (100) with it.

Any appropriate type of container (102) may be used to hold and apply the solid antiperspirant composition (100) to skin. For example, a container (102) with an oval cross section may be used to hold the solid antiperspirant composition (100). Also, a container (102) with a more cylindrical cross section may be used in some examples. The container (102) may have any appropriate shape in accordance with the principles described herein. In some examples, the container (102) may include a protective cover.

During use, a top of the container (102) may be removed and the solid antiperspirant composition (100) exposed. Via the container (102), a user may apply the solid antiperspirant composition (100) to skin by rubbing the solid antiperspirant composition (100) across the skin.

The solid antiperspirant composition (100) may include an antiperspirant agent. The antiperspirant agent may be an active ingredient that may reduce perspiration, perspiration odors, perspiration wet spots, or combinations thereof. In some examples, the antiperspirant agent may be a salt that reduces perspiration by diffusing through the sweat ducts of sweat glands. The salts may then combine with proteins to plug sweat ducts. Accordingly, perspiration cannot diffuse from the sweat glands to the surface of the skin.

Examples of antiperspirant agents include, aluminum halides, aluminum chlorohydrates, aluminum hydroxyhalides, zirconyl oxyhalides, zirconyl hydroxyhalides, aluminum dichlorohydrate, aluminum-zirconium octachlorohydrate, aluminum sesquichlorohydrate, aluminum chlorohydrex propylene glycol complex, aluminum dichlorohydrex propylene glycol complex, aluminum sesquichlorohydrex propylene glycol complex, aluminum chlorohydrex polyethylene glycol complex, aluminum dichlorohydrex polyethylene glycol complex, aluminum sesquichlorohydrex polyethylene glycol complex, aluminum-zirconium trichlorohydrate, aluminum zirconium tetrachlorohydrate, aluminum zirconium pentachlorohydrate, aluminum zirconium octachlorohydrate, aluminum zirconium trichlorohydrex glycine complex, aluminum zirconium tetrachlorohydrex glycine complex, aluminum zirconium pentachlorohydrex glycine complex, aluminum zirconium octachlorohydrex glycine complex, zirconium chlorohydrate, aluminum chloride, and combinations thereof.

While the above examples have been described with reference to specific types of antiperspirant agents, any appropriate acid or other type of agent may be used in accordance with the principles described herein. For example, other acids, such nitric acid, sulfamic acid, hydrochloric acid, and hydroxyacetic acid may be included in the solid antiperspirant composition (100). Further, the antiperspirant agent may work in conjunction with acid salts or other non-aqueous agents.

While the above examples have been described with reference to specific types of antiperspirant agents, any appropriate antiperspirant agent may be used in accordance with the principles described herein. For example, the antiperspirant agents may be used to prevent perspiration and perspiration odors, inhibit the formation of perspiration and perspiration odors, or otherwise contribute to reducing perspiration. Further, the antiperspirant agent may include multiple types of antiperspirant agents that reduce perspiration and perspiration odors. In such examples, each of the antiperspirant agents may perform different functions, perform overlapping functions, perform the same functions, or combinations thereof.

While the examples above have been described with specific reference to antiperspirant agents that are acidic, the antiperspirant agent may have any appropriate property that contributes to preventing or reducing perspiration, perspiration odors, perspiration wetness or combinations thereof in accordance with the principles described herein. For example, the antiperspirant agent may have an acidic property, an alkaline property, an abrasive property, a chemical property, a surfactant property, another type of property, or combinations thereof that contribute to preventing or reducing perspiration, perspiration odors, perspiration wetness or combinations thereof.

The solid antiperspirant composition (100) may also include an enhanced evaporative component. The enhanced evaporative component may include a spreading agent. As the surface tension of the perspiration molecules is reduced, the same volume of perspiration molecules may spread over a larger surface area. The increased surface area improves the enhanced evaporative component of the solid antiperspirant composition (100). Accordingly, the spreading agent may enhance an actual evaporation of perspiration molecules. Examples of spreading agents include alkyl-modified-trisiloxane; polysilicone-17 and trideceth-5 and meroxapol 172; and polysilicone-16 and trideceth-5. In some examples, the spreading agent may include Silsoft™ products distributed by Momentive with headquarters in Columbus, Ohio. Examples of Momentive products that may be used as spreading agents include Silsoft™ 034 (alkyl-modified-trisiloxane), Silsoft™ Spread TT (polysilicone-17 and trideceth-5 and meroxapol 172), Silsoft™ Spread SEL (polysilicone-16 and trideceth-5). In addition to improving an actual evaporation time, the spreading agent may improve application aesthetics, in other words, the “feel” of an antiperspirant as it is applied to skin. In some examples, the spreading agent may form 0.1 to 1.0 weight percent of the enhanced evaporative component (100).

The inclusion of a spreading agent to enhance an actual evaporative effect may represent a unique aspect of the solid antiperspirant composition (100) as general practice in the industry relies on an active ingredient (e.g., an antiperspirant agent), rather than a passive ingredient (e.g., a spreading agent) to enhance wetness protection. For example, the general practice in the industry has been to increase active ingredient concentrations, incorporate higher performing active ingredients, incorporate release enhancing materials such as alkoxylated alcohols, and incorporate moisture absorbing minerals to enhance wetness protection. By comparison, the solid antiperspirant composition (100) implements a surface-tension reducing spreading agent to enhance actual evaporation. In other words, the spreading agent utilizes a non-obvious mechanism to improve the evaporation of perspiration molecules.

In some examples, the enhanced evaporative component of the solid antiperspirant composition (100) may include a volatile. The volatile may increase the volatility of perspiration molecules. As used herein, volatility may refer to a measure of a tendency of a composition to vaporize. Accordingly, the volatile may increase the tendency of perspiration to vaporize, or evaporate. In other words, the volatile may provide a dry feel and reduce the sensation of perspiration. In some examples, the volatile may be 40.0 to 60.0 weight percent of the enhanced evaporative component.

In some examples, the volatile may be soluble with a surfactant that further reduces the surface tension of perspiration molecules. In another example, the volatile may be solubilized with the surfactant. A surfactant is a compound that lowers the surface tension of a liquid or the interfacial tension between two liquids or between a liquid and a solid. When added to a volatile, a surfactant significantly reduces the surface tension of the volatile allowing the volatile to penetrate the perspiration molecules rather than slide off the skin.

Surfactants have a hydrophobic end and a hydrophilic end. The hydrophobic end has an uncharged carbohydrate group that can be straight, branched, cyclic or aromatic. Depending on the nature of the hydrophilic part the surfactants are classified as anionic, nonionic, cationic or amphoteric. Anionic surfactants have a hydrophilic end that has a negatively charged group like a sulfonate, sulfate, or carboxylate and are sensitive to water hardness. Nonionic surfactants include a non-charged hydrophilic part, e.g. an ethoxylate. Nonionic surfactants are not sensitive to water hardness. Cationic surfactants have a hydrophilic end that contains a positively-charged ion. Amphoteric surfactants or Zwitterionic surfactants have both cationic and anionic centers attached to the same molecule. The surfactants in the solid antiperspirant composition (100) may include any appropriate type of mixture of surfactants. For example, the surfactants may include a blend of multiple different types of surfactants.

The volatile may have a flash point of at least 52 degrees Celsius. As used herein, the term flash point may be the lowest temperature at which the volatile can vaporize. Such a flash point may allow the volatile to be included in molten materials such as an invisible solid antiperspirant composition (100). Examples of volatiles include cyclic siloxanes, linear siloxanes, isoparaffins, alkanes, alcohols, or combinations thereof. As described above, the inclusion of a volatile may be beneficial in that it delivers a dry, and potentially comfortable, feel to the antiperspirant application, and delivers an enhanced perceived evaporation. Both of which may result in a more satisfactory consumer experience. This may be a non-obvious aspect in that previous attempts to increase evaporative effects has focused on the active ingredient, not an passive ingredient such as the enhanced evaporative component, and more specifically a volatile.

In addition to the first spreading agent and the volatile, the enhanced evaporative component may include a second spreading agent to increase the solvency of the first spreading agent. Examples of the second spreading agent include alcohols, glycols, sorbitols, glycerols, or combinations thereof. Specifically, the second spreading agent may be the Abil® B 8843 product (PEG-14 dimethicone) distributed by Evonik industries with headquarters in Germany. Adding a second spreading agent may increase the transportation of perspiration away from the skin through increased evaporation. In some examples, the second spreading agent may be 1.0 to 10.0 weight percent of the enhanced evaporative component.

The enhanced evaporative component of the solid antiperspirant composition (100) may also include an entrapment material to contain the volatile. The entrapment material may regulate the release of the volatile over a period of time. Accordingly, the entrapment material may offer continual release of the volatile. Accordingly the perceived sensation of increased wetness protection may be prolonged using the entrapment material. The entrapment material may be a material that can absorb, adsorb, or encapsulate the volatile. Examples of entrapment materials include starches, gelatins, polycapsulates, xeolites, silicas, or combinations thereof.

The entrapment material may be configured to break down, and thus release the volatile. In one example, the entrapment material may break down when in contact with perspiration molecules. In other words, the entrapment material in the solid antiperspirant composition (100) may be applied to skin. As the entrapment material comes into contact with perspiration, the housing of the entrapment material may break down, releasing the volatile to react with perspiration molecules.

In another example, the entrapment material may be configured to break down, and release the volatile, through a physical rupture of the entrapment material housing, for example, when the solid antiperspirant composition (100) is rubbed on skin via the container (102). Using the entrapment material may be beneficial in that it provides a time release of the volatile, and accordingly increases the duration of the dry “feel” provided by the volatile. Accordingly, the enhanced wetness protection exhibited by the solid antiperspirant composition (100) is perceived to be over a longer period of time. In some examples, the entrapment material may form 50.0 to 75.0 weight percent of the enhanced evaporative component.

While the examples above have been described with reference to specific materials for controlling the release of the volatile, any appropriate material for controlling how and when the volatile may be used in accordance with the principles described herein. For example, some of the materials described above may be used to cause some amount of the volatile to be released immediately while other materials are modified to delay a release of the volatile or to slow the release of the volatile. Such timing can provide for a more continuous release of the volatile to provide a more continuous exposure to the perspiration while the volatile is most effective.

In addition to the elements described above, the solid antiperspirant composition (100) may include other ingredients. For example, the solid antiperspirant composition (100) may include anti-bacterial additives, dyes, antioxidants, and moisturizers, among other additives to achieve a desired purpose or function.

Specifically, fragrances may be included in the solid antiperspirant composition (100) to provide the solid antiperspirant composition (100) with a pleasant smell. A non-exhaustive list of fragrances that may be used in the solid antiperspirant composition (100) include triclosan, triclocarban, usnic acid salts, zinc phenolsulfonate, b-chloro-D-alanine, D-cycloserine, animooxyacetic acid, cyclodextrine, sodium bicarbonate, and combinations thereof.

Further, the solid antiperspirant composition (100) may include at least one pigment. A pigment may be any composition that changes the color of the solid antiperspirant composition (100). The pigments may include any appropriate type of pigment according to the principles described herein. For example, the pigments may include cadmium, carbon, chromium, copper, iron, clay, titanium, another element, or combinations thereof.

The solid antiperspirant composition (100) may include a deodorant to reduce odors emanating from perspiration. Examples of deodorant additives include any agent to neutralize, suppress, or mask perspiration odor.

The solid antiperspirant composition (100) may include the following ingredients in the corresponding weight percent ranges as indicated in Table (1):

TABLE (1)

| Ingredient | Range (weight percent) |
|---|---|
| Cyclopentasiloxane | 32.0-46.0 |
| Stearyl Alcohol | 15.0-24.0 |
| Aluminum Zirconium Trichlorohydrex | 5.0-25.0 |
| PPG-14 Butyl Ether | 7.0-15.0 |
| Hydrogenated Castor Oil | 1.0-8.0 |
| Myristal Myristate | 1.0-8.0 |
| Enhanced Evaporative Component | 0.1-6.0 |

In Table (1), the enhanced evaporative component may include a first spreading agent, the volatile, a second spreading agent, and the entrapment material. In some examples, the enhanced evaporative component may include a spreading agent, a volatile, a second spreading agent, and the entrapment material may be in weight percent ranges as indicted in Table (2).

TABLE (2)

| Ingredient | Range (weight percent) |
|---|---|
| First Spreading Agent (Silsoft ™ 034) | 0.1-1.0 |
| Volatile (Cyclotetrasiloxane) | 40.0-60.0 |
| Second Spreading Agent (Abil ® B 8843) | 1.0-10.0 |
| Entrapment Material (Zea mays corn starch) | 50.0-75.0 |

While Table (2) describes specific chemicals with specific weight percentages, Table (2) is an example, and any appropriate chemical at any appropriate weight percent may be used in accordance with the principles described herein.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A solid antiperspirant composition with an enhanced evaporative component, comprising:
an antiperspirant agent disposed within the solid antiperspirant composition; and
an enhanced evaporative component disposed within the solid antiperspirant composition, the enhanced evaporative component comprising:
a first spreading agent configured to reduce the surface tension of perspiration molecules;
a volatile configured to increase the volatility of perspiration molecules; and
an entrapment material to contain the volatile.

2. The solid antiperspirant composition of claim 1, wherein the antiperspirant agent is configured to reduce perspiration, perspiration odors, perspiration wetness, or combinations thereof.

3. The solid antiperspirant composition of claim 1, wherein the first spreading agent forms 0.1 to 1.0 weight percent of the enhanced evaporative component.

4. The solid antiperspirant composition of claim 1, wherein the first spreading agent comprises alkyl-modified-trisiloxane; a mixture comprising polysilicone-17, trideceth-5, and meroxapol 172; a mixture comprising polysilicone-16 and trideceth-5; or combinations thereof.

5. The solid antiperspirant composition of claim 1, wherein the volatile comprises a siloxane, an isoparaffin, an alkane, an alcohol, or combinations thereof.

6. The solid antiperspirant composition of claim 1, in which the entrapment material comprises a starch, a gelatin, a polycapsulate, a xeolite, a silica, or combinations thereof.

7. The solid antiperspirant composition of claim 1, in which the entrapment material is configured to breakdown when put in contact with perspiration molecules.

8. The solid antiperspirant composition of claim 1, in which the entrapment material is configured to physically rupture.

9. The solid antiperspirant composition of claim 1, in which the entrapment material forms 50.0 to 75.0 weight percent of the enhanced evaporative component.

10. The solid antiperspirant composition of claim 1, further comprising a second spreading agent to increase the solvency of the first spreading agent, wherein the second spreading agent forms 1.0 to 10.0 weight percent of the enhanced evaporative component.

11. An antiperspirant product with an enhanced evaporative component, comprising:
a container; and
a solid antiperspirant composition housed within the container, wherein the solid antiperspirant composition includes:
an antiperspirant agent;
an enhanced evaporative component comprising:
a spreading agent configured to increase an evaporation of perspiration molecules;
a volatile configured to increase the volatility of perspiration molecules; and
an entrapment material to contain the volatile.

12. The antiperspirant product of claim 11, wherein the antiperspirant agent is configured to reduce perspiration, perspiration odors, perspiration wetness, or combinations thereof.

13. The antiperspirant product of claim 11, wherein the container comprises a dispenser to push the solid antiperspirant composition out of the container.

14. The antiperspirant product of claim 11, wherein the spreading agent comprises a composition of multiple spreading agents.

15. The antiperspirant product of claim 11, wherein the spreading agent forms 0.1 to 1.0 weight percent of the enhanced evaporative component.

16. The solid antiperspirant composition of claim 11, wherein the spreading agent forms 0.1 to 10.0 weight percent of the enhanced evaporative component.

17. The antiperspirant product of claim 11, wherein the first spreading agent comprises alkyl-modified-trisiloxane; a mixture comprising polysilicone-17, trideceth-5, and meroxapol 172; a mixture comprising polysilicone-16 and trideceth-5; an alcohol, a glycol, a sorbitol, a glycerol, or combinations thereof or combinations thereof.

18. The antiperspirant product of claim 10, further comprising a surfactant soluble with the volatile.

19. The antiperspirant product of claim 10, wherein the volatile forms 40.0 to 60.0 weight percent of the enhanced evaporative component.

20. A solid antiperspirant composition with an enhanced evaporative component, comprising:
an active ingredient comprising an antiperspirant agent to reduce perspiration, perspiration odors, perspiration wetness, or combinations thereof;
a passive ingredient comprising an enhanced evaporative component comprising:
a spreading agent forming 0.1 to 10.0 weight percent of the enhanced evaporative component; and
a volatile forming 40.0 to 60.0 weight percent of the enhanced evaporative component; and
an entrapment material to contain the volatile.
